(19) United States
(12) Patent Application Publication
(10) Pub. No.: US 2023/0116817 A1
(43) Pub. Date: Apr. 13, 2023

(54) DEVICE AND METHOD FOR DETECTION OF VIRUSES BY XRF

(71) Applicant: SECURITY MATTERS LTD., Kibbutz Ketura (IL)

(72) Inventors: Yifat BAREKET, Reut (IL); Tehila NAHUM, Holon (IL); Michal FIRSTENBERG, Rehovot (IL); Nataly TAL, Nes Ziona (IL); Mor KAPINSKY, Herzeliya (IL); Hagit SADE, Ramat-Gan (IL); Dana GASPER, Tel Aviv (IL); Haggai ALON, Kibbutz Naan (IL); Ron DAFNI, Tel Aviv (IL); Chen NACHMIAS, Tel Aviv (IL); Gal SHMUELI, Or Yehuda (IL); Avital TRACHTMAN, Lod (IL); Yonatan MUSNIKOW, Kibbutz Ketura (IL); Maria CHUCHAEV, Kibbutz Yahel (IL); Nadav YORAN, Tel Aviv (IL)

(21) Appl. No.: 17/907,334

(22) PCT Filed: Mar. 24, 2021
(86) PCT No.: PCT/IL2021/050325

§ 371 Date: Sep. 26, 2022

Related U.S. Application Data
(60) Provisional application No. 63/000,277, filed on Mar. 26, 2020.

(51) Int. Cl. G01N 33/569; G01N 33/543

(52) U.S. Cl. CPC G01N 33/56983; G01N 33/54386

(57) ABSTRACT

The invention provides methods and tools for the directed and indirect detection of infection with microorganisms pathogens in biological and non- biological samples, and specifically applications of XRF (X-ray fluorescence) methodology for the detection of infections with viral and bacterial pathogens responsible for the widespread epidemics in mammals and humans, including the current pandemic of COVID-19.

### FIELD OF THE INVENTION

The present invention generally relates to methods and tools for the directed and indirect detection of infection with microorganisms pathogens in biological and non-biological samples, and specifically applications of XRF (X-ray fluorescence) methodology for the detection of infections with viral and bacterial pathogens responsible for the widespread epidemics in mammals and humans, including the current pandemic of COVID-19.

### BACKGROUND

The current Coronavirus Disease (COVID-19) has awakened our persisting concerns about worldwide epidemics. Only in the span of the 20^{th} and 21^{st} centuries, the humanity had to face a significant number of perilous epidemics: the Spanish influenza in the beginning of the 20^{th} century, the polio epidemic in the early 50s, AIDS in the late 80^{th}, Avian influenza (H5N1) in the late 20^{th} century, Severe Acute Respiratory Syndrome (SARS) in 2002, Ebola between 2013 and 2016, and COVID 19 (SARS-CoV-2) today. Some of them remained confined to endemic populations and regions, and the others were widespread in many parts and world populations.

The general view is that epidemics and pandemics with viral and bacterial pathogens are an integral part of our way of life, increasing population growth, clustering in major metropolitan areas, globalization, and others. Epidemics and pandemics are, therefore, here to stay and will keep on following us in the future.

Therefore, rapid detection of the biological cause of an emerging or ongoing epidemic, being it viral or bacterial, and prevention of its propagation in populations that are naive or previously unexposed to the cause, are one of the biggest challenges the society faces today.

The current epidemic of COVID-19 is one such example. COVID-19 emerged in the late 2019 and the beginning of 2020, initially in Wuhan, Hubei province, China. It is likely that the disease has originated in bats and was transmitted to humans through yet unknown secondary intermediary host. Early on it became apparent that the disease is predominantly transmitted by inhalation or contact with the causative agent of COVID-19, the SARS-CoV-2 virus, by contact with contaminated surfaces or lung and nasal discharge of infected patients with the incubation period from 2 to 14 days.

It is now known that SARS-CoV-2 is also present in the saliva of infected patients, and that the viral levels in saliva are strongly indicative of a severe disease.

Asymptomatic patients pose a significant concern. Now, evidence suggests that about 1 in 5 infected people experience no symptoms. And while this is a relief for the presently occupied hospitals and healthcare facilities, researchers are still divided about whether asymptomatic infections are acting as a ‘silent driver’ of the pandemic.

A number of laboratory tests are available for the detection of SARS-CoV-2. Being a positive-sense single-stranded RNA, most tests are based on some kind of nucleic acid amplification in nasopharyngeal swabs (e.g., reverse transcription polymerase chain reaction (RT-PCR), transcription-mediated amplification (TMA), or loop-mediated isothermal amplification). Detection of a past infection is possible with serological tests.

Therefore, efficient and sensitive testing for SARS-CoV-2, through the entire range of its emerging variants, remains the primary means of managing and controlling the COVID-19 infection on the regional and state levels in populations worldwide.

The inventors have previously described certain applications of X-ray fluorescence (XRF) technology for marking and detecting certain non-biological materials and objects (see US 2018/0095045).

### GENERAL DESCRIPTION

The present invention provides a rapid, high throughput, highly sensitive and convenient methods and tools and for the detection of a wide range of biological pathogens, and specifically the airborne or waterborne bacteria and viruses that are responsible for a major part of world epidemics.

At the core of the invention is X-ray fluorescence (XRF) marking technology used to detect and possibly quantify chemical material elements and/or composition constituents characteristic of an object. Reading XRF signals is indicative of materials and compositions and can be used for marking and detecting objects.

XRF is the emission of characteristic ‘secondary’ (or fluorescent) X-rays from a material that has been excited by a bombardment with high-energy X or gamma rays. The phenomenon is widely used for elemental or chemical analysis, particularly in the investigation of metals, glass, ceramics and building materials, in geochemistry, forensic science, archaeology, and art objects. It has been rarely applied to biological materials.

The present invention offers an application of XRF detection for bio-medical purposes, whereby the XRF identifiable markers, methods and the respective reading device are adapted to include additional functional elements to provide a highly sensitive, specific, and rapid molecular diagnostic tool to be used for the detection of various biological pathogens in various clinical specimens. Due to its excellent trace element sensitivity and large penetration depth, this technique does not require fractionation or sectioning, and permits to analyze and quantify XRF identifiable markers in whole samples, close to their natural origin.

More specifically, according to the invention the XRF identifiable markers are operable in conjunction with various technologies for direct or indirect molecular detection of biological pathogens. Numerous high-quality approaches have been developed so far in the context of molecular diagnostics of pathogens, including genome-based and protein-based methods for direct detection of pathogens and serological detection of host antibodies against pathogens. And despite the existing experience with such technologies, some open questions remain as to the overall specificity, sensitivity, and affordability of such tests, especially in high throughput screening applications.

Thus, in the broadest sense, the invention provides a diagnostic platform for the detection of a biological pathogen, either bacterial or viral, in populations at risk or populations already infected with the pathogen. The invention permits a high degree of flexibility and versatility, whereby a multipurpose or multifunctional element such as an XRF identifiable marker is associated or linked to a functional element or component of a genome-based or a protein-based molecular assay, so that upon reaction with the pathogen, the entire construct is identifiable by an XRF-based device.

For example, the XRF identifiable marker can be one or more atoms emitting X-rays in response to X or gamma ray radiation, which can be either metal ion(s), organometallic- or metal oxide-functionality, or non-metal atom(s). The functional component of a molecular assay can be one or more types of nucleotides in polymerase chain reaction (PCR) or reverse transcription, or one or more types of amino acids of a specific antibody or of a secondary universal antibody included in the same test.

The nature of association or linking between these two components can vary depending on the type of markers and functional components. Specific examples are covalent bonding or bonding via one or more non-specific multifunctional (or universal) entities. The specific orientation and bonding of the components can be tailored to include more than one type of marker and more than one type of functional component to enhance the sensitivity and specificity of detection of a given pathogen.

Importantly, the entire construct is operable and stable at room temperature, and the specific XRF signature of the marked pathogen is identifiable by a convenient and portable XRF-based device. At its core, the technology is non-invasive, and thus opens a broad range of potential applications.

This new diagnostic platform can be applied to a wide variety of biological specimens obtained from individuals at risk to be infected or already infected by a given pathogen, such as samples of blood, serum, saliva and sample of discharge from nasal and lung mucosa, fecal and urine samples, etc. In other words, it can be incorporated into the diagnostic testing routines for many types of pathogens, bacteria and viruses.

According to the WHO and the ECDC PHE (European Centre for Disease Prevention and Control Public Health Emergency), the epidemics or pandemics that remain relevant in the 21^{st} century include several old diseases such as cholera, plague and yellow fever, as well as emerging diseases such as severe acute respiratory syndrome (SARS), Ebola, Zika, Middle East respiratory syndrome (MERS), HIV (technically endemic), influenza A (H1N1)pdm/09 and most recently COVID-19. Tuberculosis (TB) remains the top infectious disease killer caused by a single organism and was responsible for 1.5 million deaths in 2018.

It is apparent that this list is heterogenic. It includes bacterial (e.g., cholera, TB) as well as viral (e.g., SARS, HIV, Ebola, and COVID-19) pathogens, airborne and waterborne pathogens, and pathogens that are transmitted via direct contact with infected body fluids (e.g., blood, respiratory discharges) and contaminated objects. Many of these pathogens affect the lung (e.g., SARS, TB, Influenza A and COVID-19.

The diagnostic platform of the invention is applicable to all these pathogens as an individual testing tool, and moreover, as a large-scale screening tool in populations at risk and those already infected with the pathogen.

It is also applicable for research and veterinarian purposes. For example, many of the pathogens in the above list originate from animals (e.g., Ebola, Influenza A, SARS, and more recently COVID-19).

### DETAILED DESCRIPTION

Thus, in the most general sense the present invention can be articulated in terms of diagnostic solutions for the detection of biological pathogens responsible for the recent and current epidemics in humans, and potential future epidemics caused by variants of these pathogens. All the evidence suggests that even with the successful implementation of respective vaccines, the known pathogens will continue existing in the world population or at least in certain geographical and socioeconomic pockets and can re-emerge as variants in future pandemics.

In one of its main aspects the invention provides a diagnostic platform for detecting a biological pathogen in a biological or a non-biological sample, which comprises at least one XRF identifiable marker operably linked to at least one functional component for detecting the biological pathogen or a part thereof, for marking the pathogen in the sample with the at least one XRF identifiable marker that is detectable by an XRF reading device, as further detailed herein.

The term ‘XRF’ (X-ray fluorescence) refers herein to a non-destructive analytical technique used to determine the elemental composition of materials. It further implies XRF analyzers or devices determining the chemistry of a sample by measuring the fluorescent (or secondary) X-ray emitted from a sample when it is excited by a primary X-ray source. The X-ray fluorescence analysis is a method that uses characteristic X-rays (fluorescent X-rays) generated when X-rays irradiate a substance.

Therefore, the XRF identifiable marker is any marker that is detectable by an XRF analyzer or device (also XRF reader).

In numerous embodiments the XRF identifiable marker of the invention is an atom or a group of atoms emitting an X-Ray signal in response to X or gamma ray radiation.

X-rays encompass herein electromagnetic waves comparable to visible light rays but with an extremely short wavelength that measures from 100 A to 0.1 A. It further encompasses rays that easily pass-through substances and become stronger as the atomic number of a substance through which it passes decreases.

In certain embodiments the XRF identifiable marker is a metal ion, an organometallic functionality, a metal oxide functionality or a non-metal atom.

Examples of metal ions include, but not limited to, spectroscopically silent metal ions like potassium, sodium, calcium, magnesium, and zinc, together with the more spectroscopically accessible iron, copper, manganese, and a few others.

Organometallic compounds encompass herein any compound containing carbon atoms bonded to a metal, including covalently bound compositions. It further includes organometallic complexes between a metal and an organic ligand and ionic bonded organometallic compounds like alkali, alkaline earth metals, Lanthanides and Actinides.

Metal oxides refers herein metal oxides and mixed meta oxides, with specific examples of iron, silicon, titanium, and aluminium oxide that are further distinguished by the numbers of oxygen atoms involved and the element’s oxidation number.

In further embodiments wherein the XRF identifiable marker is an element or a functional group comprising an element selected from Si, P, S, Cl, K, Ca, Br, Ti, Fe, V, Cr, Mn, Co, Ni, Ga, As, Fe, Cu, Zn, Ga, Rb, Sr, Y, Zr, Nb, Mo, Tc, Ru, Rh, Ag, Cd, In, Sn, Sb, Te, I, Cs, Ba, La, Ta, W, Se and Ce.

The term ‘functional component for detecting the biological pathogen or a part thereof implies herein a specific reaction between the functional component and the biological pathogen, whereby the biological pathogen can be detected, identified and/or amplified. In other words, the reaction between the functional component and the biological pathogen implies a molecular assay that detects one or more markers, structures or sequences in the genome or proteome of the biological pathogen.

In numerous embodiments the at least one functional component is a component of a polymerase chain reaction (PCR), a reverse transcription, a nucleic acid hybridization, an antibody test, a serological antibody test

PCR is an example of a molecular assay enabling targeted amplification and individuation of specific genomic sequences of the pathogen. PCR encompasses herein a wide range of technologies that produce multiple copies of a specific DNA region in vitro (amplicon), which include but not limited to the classic 3-step PCR (denaturation of the template; annealing of primers; and extension of the new DNA strands), Real-Time PCR (quantitative PCR or qPCR), Reverse-Transcriptase (RT-PCR); Multiplex PCR, Nested PCR, High Fidelity PCR, Fast PCR, Hot Start PCR, GC-Rich PCR.

Reverse transcription is a specific example of PCR for amplification and individuation of RNA sequences using a prior step with reverse transcriptase to covert RNA to DNA. This type of detection assay is particularly applicable to RNA viruses (e.g., SARS-CoV-2, Influenza).

Nucleic acid hybridization is another type of a genomic detection assay targeting both DNA or RNA sequences of the pathogen. Nucleic acid hybridization encompasses herein any length of target DNA or RNA sequences and any length of DNA or RNA probes, as short-stretch oligonucleotides (e.g., 21-base) and longer stretches, and derivatives.

An antibody test is an example of a molecular assay enabling the detection of specific peptides or proteins characteristic of a given pathogen. Antibody test encompasses herein priory and secondary antibodies, when the latter are used in conjunction with specific antibodies, and further antibodies produced for proteins on the surface of the pathogen (e.g., viral envelope proteins, viral spike proteins) and proteins from the internal milieu of the pathogen. It further encompasses the antibodies produced to secondary modifications of such proteins.

Serological antibody test (or serological test) is an example of a molecular assay enabling the detection of specific antibodies produced in the host in response to the biological pathogen. Serological test encompasses herein different types of serological tests, such as flocculation, neutralization, agglutination, precipitation, complement fixation and enzyme-linked immunosorbent assays (ELISAs). It further encompasses the IgG and the IgM types of host antibodies produced indifferent infection windows.

In certain embodiments the at least one functional component can be a nucleotide, an oligonucleotide, an amino acid, a peptide, a derivative, a modification, a fusion construct, a conjugate thereof.

In certain embodiments the functional component can be a nucleotide or a polynucleotide (primer) employed in a PCR for targeted amplification and individuation of specific DNA sequences of a given pathogen (e.g., a Vibrio cholerae).

In other embodiments the functional component can be a nucleotide or a polynucleotide (primer) employed in a RT-PCR for targeted amplification and individuation of specific RNA sequences of a given pathogen (e.g., SARS-CoV-2).

In still other embodiments the functional component can be a nucleotide or a polynucleotide (oligonucleotide) employed in nucleic acid hybridization for the detection of specific DNA or RNA sequences of a given pathogen.

In other embodiments the functional component can be an amino acid, a peptide, or a part of an antibody employed in the detection of specific proteins of a given pathogen.

In other embodiments the functional component can be an amino acid, a peptide, or a part of an antigen characteristic of a given pathogen, which is employed in the detection of specific IgG or IgM antibodies produced in a host against a given pathogen.

In a broader sense, the functional component can be an amino acid, a peptide, or a part of an antigen characteristic of a given pathogen, which is employed in the detection of any specific component of host immunity produced against a given pathogen.

In certain embodiments the functional component can be a derivative, a modification, a fusion construct, a conjugate of the afore-mentioned types of functional components.

Importantly, in numerous embodiment the at least one XRF identifiable marker and the at least one functional component are operably linked. The term ‘operably linked’ implies herein any type of chemical bonding that is stable at room temperature and which does not interfere with the XRF detection.

In numerous embodiments the at least one XRF identifiable marker and the at least one functional component are linked by covalent bond.

In certain embodiments the at least one XRF identifiable marker and the at least one functional component are operably linked by at least one non-specific (multifunctional) component. The term ‘non-specific component’ (also a multifunctional component) implies a universal chemical linker bridging between the XRF marker and the functional component.

In numerous embodiments the non-specific component or the chemical linker can be a linking atom or a group of atoms linking the at least one XRF identifiable marker and/or at least one functional component.

In further embodiments the non-specific component or the chemical linker can be selected from carbon-based groups, such as aliphatic groups, cyclic groups, groups containing one or more double or triple double binds, aromatic groups, heteroaromatic groups, carbocyclic groups, silicon-based groups, sugars, amino acids, nucleic acids, phosphate groups and others.

In further embodiments the non-specific component or the chemical linker is bound to the at least one XRF identifiable marker and/or at least one functional component by covalent bond.

In certain embodiments the entire complex can comprise more than one XRF identifiable marker, functional component and/or non-specific component.

In numerous embodiments the diagnostic platform of the invention can be schematically described as a structure of components A-L-B, wherein A is a pathogen binding functionality or functionalities, B is an XRF identifiable marker or markers, and L is linker or linkers associating A and B.

With respect to the relevant applications of the diagnostic platform of the invention, in numerous embodiments the relevant pathogens are viruses and bacteria associated with the main epidemics and pandemic in humans. In many cases the human host is a secondary, a tertiary or a tertiary host the same or akin pathogen, whereas the original pathogen variant was originated in another species, such as rats (e.g., Plague), chickens (e.g., Influenza), monkeys (e.g., HIV/AIS) and bats (e.g., SARS and COVID-19). For the major part, the disease in the original mammalian host is asymptomatic.

Therefore, in numerous embodiments the relevant pathogens are viral and bacterial pathogens in mammalian diseases analogous to the main epidemics and pandemic in humans.

In numerous embodiments the viral or the bacterial pathogen is selected from the pathogens of Cholera, Plague and Yellow Fever, Severe Acute Respiratory Syndrome (SARS), Ebola, Zika, Middle East Respiratory Ryndrome (MERS), Human Immunodeficiency Virus (HIV/AIDS), Influenza A (H1N1)pdm/09, Tuberculosis (TB) and Corona Virus Disease 19 (COVID-19).

Cholera herein encompasses the entire range of partial and complete symptoms of infections the small intestine caused by certain bacterial strains of Vibrio cholerae. Several types of Vibrio cholerae have been related to the disease, with some types producing more severe symptoms than others. Cholera encompasses herein Cluster I and Cluster II strains related to the past infections, and also more recent strains from the African continent and Yemen. Cholera is predominantly transmitted by contaminated water and food.

Plague herein encompasses the entire range of infectious diseases caused by the bacterium Yersinia pestis. It encompasses bubonic and septicemic plague transmitted by flea bites or handling infected animals, and further the pneumonic form transmitted through the air via infectious droplets.

Yellow Fever encompasses herein the entire range of symptoms of acute viral haemorrhagic disease caused by an arthropod borne Yellow fever virus (YFV) belonging to the family Flaviviridae. YFV is endemic to South American countries and much of sub-Saharan Africa.

SARS encompasses herein the entire range of symptoms of a viral respiratory disease caused by a SARS-associated coronavirus. SARS is an airborne virus and can spread through small droplets of saliva, and nasal and lung discharge. It has a capacity to spread via surfaces and routes of international air travel. Corona viruses, e.g., SARS MERS and COVID-19, have positive-sense RNA genome.

Ebola, also Ebola hemorrhagic fever (EHF), encompasses herein the entire range of symptoms of infection caused by certain species of Ebola viruses, including the species of Zaire ebolavirus, Sudan ebolavirus, Taï Forest ebolavirus, Côte d'Ivoire ebolavirus, Bundibugyo ebolavirus, and further species that cause analogous disease in a mammalian host. Ebola is a single stranded RNA virus.

Zika encompasses herein the entire range of symptoms of infection caused by infection is caused by an arthropod borne Zika virus belonging to the family Flaviviridae. Zika is endemic to South American countries.

MERS refers herein to a viral respiratory disease similar to SARS, which is caused by another type of corona virus.

HIV/AIDS encompasses herein the entire range of symptoms of infection caused by retroviruses HIV-1 and HIV-2, including multiple strains and sub-strains of these viruses identified so far.

Influenza A encompasses herein the entire range of symptoms of infection caused by an influenza virus, the only species of the genus Alpha-Influenzavirus of the virus family Orthomyxoviridae, including the strains and sub-types isolated from humans and domestic birds. Influenza has fragmented RNA genome (8 segments).

TB herein encompasses the entire range of respiratory symptoms caused by Mycobacterium tuberculosis (MTB) bacteria, including various types of TB such as latent and including multi-drug resistant TB and further, more recent MTB isolates from Chine, Africa, Russia and Latin America.

COVID-19 herein encompasses the entire range of respiratory and other symptoms of infection caused by the new corona virus SARS-CoV-2, including the genetic variants isolated so far.

In numerous embodiments the diagnostic platform of the invention can be applied to individuals who are infected with one of the above viruses or bacteria and who present partial or more complete clinical manifestations of the respective disorders, or infected individuals with severe or mild symptoms related to these disorders.

Yet in other embodiments the diagnostic platform of the invention can be applied to infected individuals who are asymptomatic and who do not present any symptoms related to the above disorders.

Still in other embodiments the diagnostic platform of the invention can be applied to individuals who are risk of being infected with one of the viruses and bacteria and who seek negative diagnosis of the respective infection.

These two latter applications are particularly important. Separation and distancing of infected and non-infected population groups is one of the key measures to control the spread of epidemics.

With respect to the tested material, in numerous embodiments the diagnostic platform of the invention is applied to a biological sample obtained from an individual at risk of being infected or already infected with the biological pathogen.

In certain embodiments the diagnostic platform of the invention is applied to a sample of a mammal.

In numerous embodiments the diagnostic platform is applied to a sample of a body fluid, a body discharge, a body secretion obtained from a mammal at risk of being infected or already infected with the biological pathogen.

In further embodiments the diagnostic platform is applied to a sample of blood, serum, saliva, a sample of nasal or lung discharge, urine or fecal sample or a sample of tear drops.

Yet in other embodiments the diagnostic platform can be applied to a non-biological sample obtained from a surface, an object, or a part thereof, which was in contact or is suspected to be in contact with a mammal at risk of being infected or already infected with the biological pathogen. This application is important to exclude transmission of the pathogen from contaminated surfaces or objects

In numerous embodiments the diagnostic platform of the invention is operable at room temperature.

In numerous embodiments the diagnostic platform of the invention is operable with an XRF reading device for the detection of specific XRF signature of the pathogen.

In certain embodiments, said device is a portable XRF reading device.

The invention can be further articulated in a form of composition and methods using the above diagnostic platform, through the entire rage of its specific embodiments.

More specifically, the compositions and methods of the invention comprise or involve administration of at least one XRF identifiable marker operably linked to at least one functional component for detecting a biological pathogen or a part thereof, thereby marking the pathogen with the at least one XRF identifiable marker that is detectable by an XRF reading device.

In numerous embodiments the compositions and methods of the invention use XRF identifiable marker that is an atom or a group of atoms emitting an X-Ray signal in response to X or gamma ray radiation.

In numerous the embodiments the compositions and methods of the invention use an XRF reading device for the detection of specific XRF signature of the pathogen.

Other specific embodiments of the XRF marker-functional component construct, modification of its specific components, and its relevant applications have been discussed above.

Specifically on the compositions of the invention, in numerous embodiments the compositions can further comprise at least one additive selected from stabilizers, antioxidants, oxidizing materials, surfactants, lysing agents, sugars, salts, pH stabilizers, buffers, detergents, diluents, preservatives, solubilizers and emulsifiers.

Specifically on the methods of the invention, an obligatory step is contacting a sample with a potential biological pathogen with at least one XRF identifiable marker operably linked to at least one functional component for detecting the biological pathogen or a part thereof, thereby marking the pathogen in the sample with the at least one XRF identifiable marker that is detectable by an XRF reading device.

Once the sample is treated with the functional component, detection by XRF may progress. Thus, in methods of the invention, e.g., for determining the presence of a virus in a sample, the method comprises irradiating said sample with X-ray or gamma-ray radiation to thereby determine presence of the pathogen, e.g., a virus. The XRF unit may be any unit known in the art. XRF units and detection methods which may be used according to the invention are disclosed, for example, in U.S. Pat. Nos. 10,607,049, 10,539,521 and International Applications Nos. PCT/IL2017/050121, PCT/IL2017/050354, PCT/IL2017/051050 and any US counterpart application, each of which being herein incorporated by reference.

In numerous embodiments the methods of the invention can further include the step of incubating the sample with the at least one XRF identifiable marker and the at least one functional component.

In other embodiments the methods of the invention can further include the step of removing an excess of the at least one XRF identifiable marker and the at least one functional component from the sample.

Yet in other embodiments the methods of the invention can further include the step of lysing the sample before contacting the sample with at least one XRF identifiable marker operably linked to at least one functional component.

It is another important aspect of the invention to provide a kit comprising the diagnostic platform or the composition of the invention as described above, and further comprising instructions for use.

A kit implies a predetermined amount and distribution of respective components and the distribution thereof in appropriate packages or containers.

Ultimately the invention can be articulated in the form of use of the diagnostic platform or the composition of the invention as described above for detecting or excluding the presence of a biological pathogen in a biological and non-biological sample.

### Specific Embodiments

In one of its main aspects the invention provides a diagnostic tool for diagnosing/determining presence of a virus in a biological sample, the tool being in a form of a multifunctional material having at least one functionality selected and operable to associate to at least one region of a virus and at least one functionality identifiable by XRF.

In numerous embodiments the tool of the invention is applied to the biological sample that is a sample suspected of containing a viral pathogen.

The viral envelope consists of a lipid bilayer where the membrane, envelope and spike structural proteins are anchored. A subset of coronaviruses also have a shorter spike-like surface protein called hemagglutinin esterase (HE). Some viruses have viral envelopes which have a protein layer, a capsid, which separates the envelope and the virus genome. The virus envelopes are typically comprises of phospholipids and proteins and may include viral glycoproteins.

Inside the envelope, the nucleocapsid is formed of multiple copies of a nucleocapsid protein that is bound to the positive-sense single-stranded RNA genome in a continuous beads-on-a-string type conformation.

The inventors of the invention disclosed herein have developed a facile and fast kit for diagnosing the presence of viruses in a biological sample. The method utilizes a material having the capability of selectively binding to a virus and system for identifying, recording and analyzing such a binding event by x-ray fluorescence (XRF).

Thus, in a first aspect of the invention, there is provided a diagnosing tool for diagnosing/determining presence of a virus in a biological sample, the tool being in a form of a multifunctional material having at least one functionality selected and operable to associate to at least one region of a virus, viral component or antibodies, cells or any other material or substance generated by an immune response to the presence of the virus, and at least one functionality identifiable by XRF.

The virus to be identified in a biological sample is any virus species, type, subtype, or strain that may be active in one or more host species. Such a virus may be selected from coronaviridae/corona-virus, orthomyxoviridae, paramyxoviridae, Coxsackie family of viruses and adenoviridae family.

In some embodiments, the corona-virus is a COVID-19 causing pathogen, e.g., SARS-CoV-2. As known in the art, the SARS-CoV-2 encompasses SARS-CoV-2 having mutations that may be found in the entire genome of SARS-CoV-2 strains, e.g., in the 5′-UTR, ORFlab polyprotein, intergenic region, envelope protein, matrix protein, intergenic region and nucleocapsid protein.

The biological sample may be any one obtained from human or non-human subject. The sample may be a blood sample, nasal excretions, oral excretion, urine sample, tear drops and others. The biological sample may be a pre-treated sample or a sample that has not undergone any pre-treatment or manipulation. In some embodiments, the sample has undergone treatment to cause lysis of cells membrane or lysis of viruses membrane.

The biological sample may be any such sample which is suspected of containing the virus, any related viral components or any substances, antibodies or cells related to an immunogenic response to the presence of the virus in the human or animal body.

The diagnostic tool is a multifunctional material that may have any number of functionalities, at least one of which is selected and operable to associate to at least one region of a virus (so-called a virus-binding functionality) and at least one another functionality which is identifiable by XRF (so-called XRF functionality). The functionalities may be arranged around an atom or an atom-chain, e.g., a skeleton, to which the functionalities are associated. The material is typically a substantially linear material having two regions, each defining a different functionality as defined herein.

The at least one functionality that is selected to associate to a region of the virus or to viral component or antibodies, cells or any other material or substance generated by an immune response to the presence of the virus , is any such chemical functionality that can associate to an amino acid, a peptide, a protein, a lipid, a carbohydrate, a nucleic acid and other native functionalities present at said regions and components and to which association is targeted. In some embodiments, the region of the virus is a region of the membrane or the virus capsid. In other embodiments, the region is part of the virus nucleocapside.

Many types of viruses include a surface protein hemagglutinin (HA) which binding to a host cell for invading therein, neuraminidase involved in exit of virions from the host cells, M2 ion channel for balancing hydrogen ion concentrations, and ribonucleoprotein (RNP) which contains the genetic information of the virus.

The term “virus” used herein refers to an intracellular body, comprising a DNA or RNA with a nucleic code, typically a virus does not proliferate through binary fission, and does not have a particular system for generating ATP by itself. Viruses as described herein may be that which include an envelope or that which does not include an envelope (i.e., naked virus).

Generally speaking, enveloped viruses may include DNA viruses such as poxvirus, herpesvirus, and hepadnavirus; RNA viruses such as togavirus, coronavirus, hepatitis D, paramyxovirus, rhabdovirus, flavivirus, bunyavirus, orthomyxovirus ,filovirus and retrovirus. The orthomyxovirus may be selected from influenza virus A, influenza virus B, influenza virus C, isavirus, thogotovirus and quaranjavirus genera.

The coronavirus may be selected from alpha coronavirus, beta coronavirus, gamma coronavirus and delta corona virus genera. The paramyxovirus is selected from paramyxovirus, rubella virus, morbillivirus and pneumovirus genera. Further generally speaking, a virus is further comprising a nucleic acid and a protein surrounding thereof. Viral proteins are generally involved in genome protection, attachment, or fusion of virus particles to a host cell. Such proteins may include structural proteins that maintains the form of a virus and a non-structural proteins that are associated with the proteins and nucleic acids synthesis.

The term “surface proteins” in the context of the present invention is generally relates to structural proteins. Specifically, such proteins encompass all peripheral proteins that are present on an envelope of a virus. In some embodiments, such surface proteins may include a glycoprotein, and a fusion protein. In some other embodiments the surface protein may be hemagglutinin (HA), spike protein or an F protein. In some embodiments the surface proteins may be those involved in membrane fusion between the virus and target host cell.

In an embodiment, the virus is an influenza virus which includes hemagglutinin (HA).

In another embodiment of the invention, the virus is a coronavirus including spike protein as a surface protein, wherein such spike protein comprising S1 and S2 portions. Upon binding of S1 portion to a host cell, it is therefore cleaved into S1 and S2 by a protease. A hydrophobic domain at the end of S2 is exposed and is therefore activated.

In yet further embodiments, the virus is a paramyxovirus including an HN protein as a surface protein. The HN protein attaches the virus to a host cell. Such surface protein appears as a precursor HN0 in the inactive state and is being activated upon removal of an amino acid residue from the C-terminus via hydrolysis. Additionally, a paramyxovirus includes F protein as a surface protein.

The term “reaction” used herein refers to changes in physical and/or chemical states upon contact between at least one virus binding functionality and a surface protein. In some specific cases, when a surface protein get into contact with a virus binding functionality of the herein invention, such surface protein may be transformed to its active or inactive form.

The term “viral binding functionality (VBF)” as used herein refers to a molecule required for the structure, function, or signal transduction in a single cell or human or animal body. Such functionality may include a specific structure and/or region for a function or signal transduction. In some embodiments such a molecule is a ligand. The functionality may include an amino acid or a protein, a carbohydrate, fatty acids and lipids, nucleotides or nucleic acids. Also, such term may refer to any component which is able to bind to a viral portion or to any related viral components or any substances, antibodies or cells related to an immunogenic response to the presence of the virus in the human or animal body.

In some specific embodiments, the VBF is an antibody an enzyme or a ligand.

The viral binding functionality according to the invention is further linked to an XRF functionality (XF) as described herein.

Thus, provided herein is a method of determining the presence of a virus in a biological sample, the method comprising treating a biological sample with at least one diagnostic tool according to the herein invention, permitting a reaction as described herein between the VBF and the surface protein of a virus, and irradiating said sample with x-ray or gamma-ray irradiation and detecting the x-ray signal received from such sample in response to the irradiation and optionally processing the signal to thereby determine the presence of a virus.

Viral cultivation is yet another known method for detecting the availability and propagation of viral infection or a virus. In such method, susceptible cells are incubated with a material which may contain a suspected virus. After incubation, viral proteins (e.g., proteins for viral envelope and capsid) may be demonstrated in the cell. The cells are therefore treated with the diagnostic tool according to the invention, wherein the diagnostic tool comprising a VBF which is specific for viral proteins generated by the cells. Said VBF is further linked to an XRF functionality which is XRF identifiable.

Further provided is an ELISA based method for detecting a virus, any related viral components or any substances, antibodies or cells related to an immunogenic response to the presence of the virus in the human or animal body, in a sample. Such method includes introducing monoclonal antibodies which are specific against viral structures or proteins or antibodies or cells related or produced following an immunogenic response to the presence of the virus in human or animal body; such antibodies are bound to the surface of a well. Thereafter, the content of a sample in question is added to the well. If any relevant antigens are present in the added suspension, they will interact with the bound antibodies. In the next step, antigen-antibody complexations are detected by the addition of a diagnostic tool comprising VBF which is this specific embodiment is an antibody, such antibody is specific and binds to other epitopes of said viral structures or proteins. The diagnostic tool can be therefore detected by X-ray fluorescence.

Yet another option of detection which is another embodiment of the invention is by binding a virus-specific proteins to the walls of the wells, treating said wells with antibodies which are specific to the viral proteins and thereafter, treating the wells with the diagnostic tool according to the invention. Such diagnostic tool comprising VBF which is usually an antibody which is specific to the antibody utilized in the previous step to bind to the viral proteins. Usually, the binding of the VBF is to the Fc region of said antibody. Bonded antibodies can be detected by XRF as described herein.

Also provided herein is a method of detecting or determining the presence of a virus or viral infection in a sample which is based on immunofluorescence methods. Such method generally includes fixing virally infected cells to a slide and utilizing substances to increase the permeability of said cells. After, the infected and fixated cells are incubated with a suspension comprising immunoglobulins which are specific against viral proteins to be detected. The following treatment is with secondary immunoglobulins, directed against the Fc region of the previously utilized immunoglobulins and linked with an XF according to the herein invention to thereby construct a diagnostic tool. The above steps allows one to detect via XRF, viral proteins in different compartments, such as the nucleus, the cytoplasm and the cell membranes.

Also provided herein is a method of detecting viral nucleic acids to determine the presence of a virus or viral infection in a biological sample. Such method includes isolation of DNA or RNA from potentially infected cells and then cleavage of such molecules with a specific restriction enzymes. In case of DNA, the molecules are first denatured to form a single stranded strand. Subsequently, the obtained single stranded molecules are incubated with single-stranded DNA or RNA probes linked with an XRF functionality which are complementary to the nucleotide sequences examined and hybridize with them, forming double-stranded molecules. The single stranded DNA or RNA molecules (herein a virus-binding functionalities) together with the linked thereto XRF identifiable marker forming the diagnostic tool of the invention.

The present invention also describes a method of detecting or determining the presence of a virus or viral infection in a sample which is based on Polymerase Chain Reaction (PCR) or real time Polymerase Chain Reaction (rt-PCR).

Such method enables to amplify very small quantities of viral genome. The method comprising adding a primer (which acts as VBF as described) which is linked to XF, DNA polymerase and the four nucleoside triphosphates, into a mixture, to thereby enable the formation of viral complementary DNA strands. In case where the viral material is an RNA molecule, it is required to convert the RNA molecule to a DNA molecule by employing reverse transcriptase. The primer which is linked to an XF is thereby forming a diagnostic tool according to the herein invention and is detectable via the detection device as provided hereinbelow.

Such primers can be linked to an XRF identifiable marker (XF) at either the 5′ end or the 3′ end, wherein each is an embodiment of the invention.

At the end of the PCR like process, the amplified viral DNA or RNA sequences can be quantitatively detected with the detection device as described herein.

Further provided is a method of detecting or determining the presence of a virus or viral infection in a sample which is based on lateral flow immunoassay (LFIA). The method comprising a strip comprising flowable antibodies which are specific to the sample which comprises viral components or immune system components (e.g., antibodies or cells), and at least one line of fixed antibodies which are also specific to same viral/immune system components. Upon flow of a sample through the strip, antibodies which are linked to viral component will be captured by the fixed antibodies and thereby form a sandwich like structure. Flowable antibodies are further connected to an XF (usually through the Fc portion) to further construct a diagnostic tool in accordance with the invention. In case of a positive test, the fixed line will be attached to the flowable antibodies through the antigen of viral components. Since such antibodies are further linked to an XRF marker, such can be detected by a detection device as provided herein.

The term “immune system components” in accordance with the herein invention refers to any antibody (Ig), chemical substance, protein (such as cytokines) which is generated or released in response to the presence of a virus in a human or animal body.

In yet another aspect, the invention provides a method of detection as described herein. The method is for detecting viral genomic DNA or RNA molecules in a probe. Such method comprising the addition of a mixture including therein all the four nucleotides (which act herein as viral binding functionality or VBF), wherein each nucleotide is linked to an XF to thereby form a diagnostic tool in accordance with the invention.

Each of the labeled nucleotides (or the diagnostic tool) are thereby can be incorporated in a complementary DNA strand by the action of DNA polymerase.

The linkage between the VBF and the XF can be via the linker as described herein. In an embodiment, the linker is an aliphatic linker.

The XRF functionality can be linked to a nucleic acid in any portion thereof which is selected from the sugar, the base, or the phosphate group.

In some embodiments, the linkage is to the sugar.

In some embodiments, the linkage is to the base.

In some embodiments, the linkage is to the phosphate group.

Direct detection of viruses is feasible only during the acute phase of the disease, and usually not in latent or persistent infection forms. In some cases, viruses are present in the organism only before the symptomatic phase, so the direct detection of the virus is usually not successful. Therefore, infections or contact with viruses is usually detected indirectly by characterization of the developing immune response elicited against a given virus during infection. Usually, patient antibodies which specifically bind to viral proteins in the serum are detected. IgM antibodies generally indicate an acute or recent infection (e.g., viral infection). Past or former infection can be inferred by detecting IgG antibodies in the serum or a probe. Especially for diagnosis of acute infections, it is important to determine the concentration of IgM and IgG antibodies during infection. Occasionally, one also tests for IgA antibodies.

Said antibodies can be detected by any of the above means (e.g., ELISA based technique combined with an XRF marker as described), where the diagnostic tool comprising an XRF functionality which is identifiable by XRF technology or via the detection device as described hereinbelow.

SARS-CoV-2 is a single-stranded (+) RNA virus, which belongs to the genus Beta coronavirus, such virus usually comprising a spike surface glycoprotein (S), a small envelope protein (E), matrix (membrane) protein (M), and nucleocapsid protein (N). N-protein is the mostly abundant and relatively conserved protein in such viruses.

Thus, it can be conveniently utilized as diagnostic antigen for the detection by neutralization antibodies.

In coronaviruses, the S gene encodes the receptor binding spike protein, which allows receptor binding and membrane fusion. S. protein is essential for binding to host cells; it is present on the surface of virus particles and is highly immunogenic.

M and E proteins are mandatory for viral assembly. N protein is related to transcription and replication of SARS-CoV-2 RNA, and wrapping of the encapsulated genome (i.e., RNA stands) in virions. Also, the N-protein has the most acute immunogenic activity during infection. Both S and N proteins are potential antigens for detecting the virus in a sample.

The present invention provides a method for detecting viral components selected from S, E, M and N proteins via means as described above. As an example, in case a spike protein of a virus needs to be detected, an ELISA based method as described above can be utilized, where an antibody specific to the spike protein binds thereto, and where the antibody is further linked to an XRF functionality which is identifiable or detectable by XRF technology or detection device as provided herein.

Generally, IgM is the first produced antibody against viral infections. IgG is critical for long-term immunity and immunological memory. What is observed is that after corona virus infection, IgM antibodies are detected in the subject’s serum after six days and IgG after 10 days. Such antibodies persist for 2-3 years. Therefore, the detection of IgM antibodies indicates recent exposure to SARS-CoV-2, while the detection of IgG antibodies allows to determine contact tracing. Rapid detection of IgM and IgG antibodies is valuable for the diagnosis and treatment of COVID-19 disease.

Further, detection of IgA in patient’s serum is another way to provide information on the virus infection status. IgM and IgG antibodies are mostly produced against N protein of SARS and SARS-CoV-2, where IgA is produced against S1 protein of the virus. It is shown that IgA response starts even earlier than IgG response while the presence of IgG in serum continues during the infection time and can show past infections.

Therefore, provided herein is a method of determining the presence of a virus or a viral infection in a sample, the method comprising any one of the above means for detecting neutralizing antibodies selected from IgA, IgM and IgG.

In the context of the herein invention, the term “viral infection” relates to the presence of virus or viral components in a human or animal body and also relates to the presence of immune system components (including antibodies and various cells) which are produced in response to the presence of a virus or viral components in the body.

“neutralization antibodies” in the herein context are those antibodies which defends the cell from pathogens (e.g., viruses) by neutralizing any biological effects such pathogen has on the body. Such neutralization process renders the pathogen or the infectious particle to be no longer infectious. In some embodiments, said antibodies are selected from IgG, IgM and IgA.

The virus-binding functionality as defined above may bebe selected from amines such as primary, secondary and tertiary amines; sulfhydryl group, carboxylic acid and derivatives there of such as esters, amides and others; alcohols, dialcohols and higher homologs thereof; thiol, dithiols and higher homologs thereof; thio esters; nitro; ethers; di sulfides and others.

The at least one functionality which is identifiable by XRF (the so-called XRF functionality) is an atom or a group of atoms which is/are XRF sensitive in the sense that they emit an X-Ray signal in response to interrogation (irradiation) by X-Ray or gamma ray radiation. The atom or group of atoms, namely the XRF marker, may be in the form of a metal ion, an organometallic functionality, a metal oxide functionality, a non-metal atom.

In some embodiments, the XRF-marker is an element or a functional group comprising an element selected from Si, P, S, Cl, K, Ca, Br, Ti, Fe, V, Cr, Mn, Co, Ni, Ga, As, Fe, Cu, Zn, Ga, Rb, Sr, Y, Zr, Nb, Mo, Tc, Ru, Rh, Ag, Cd, In, Sn, Sb, Te, I, Cs, Ba, La, Ta, W, Se and Ce.

Where the element or atom or group of atoms is a metal atom or an ion, the XRF-functionality may be a ligand group in which the metal or ion is ligated to, by way of complexing interaction or ionic interaction. For example, the XRF-functionality may in such a case by a carboxylate anion wherein the counterion is the metal cation. In a similar way, the functionality may be a ligand moiety capable of complexing the metal atom in its uncharged form. Where the XRF-marker is a non-metal, it may be associated in the XRF-functionality via covalent bond or via a different association.

Non-limiting examples of inorganic anions include O⁻², HO⁻, F⁻, Cl⁻, Br⁻, I⁻, NO₂⁻, NO₃⁻, ClO₄⁻, SO₄⁻², SO₃⁻, PO₄⁻, PO₄⁻³, Si⁻⁴ , SiO3⁻², N3⁻, MnO₄⁻, S₂O₃⁻², SeO₄⁻², CrO4⁻², Cr₂O₇⁻² and CO₃⁻². Where the XRF-marker is a metal, the XRF functionality may be selected amongst materials comprising organic anions derived from acetates, citrates, lactates, oxalates and others.

The diagnostic tool, i.e., the multifunctional material having at least one virus-binding functionality and at least one XRF functionality, may be an organic material or an inorganic material in which the two (or more) functionalities are intimately positioned, e.g., to the same atom or same group of atoms, or may be positioned at a distance from each other along the material atom skeleton. Notwithstanding the particular arrangement, the functionality and functionality arrangement may be tailored to permit (1) facile and preferably room temperature association to the virus and (2) effective detection of the XRF marker. The diagnostic tool may thus take on the form of a carbon-chain or carbon-group associating the two or more functionalities or an inorganic material, such as a silicon-based material that associates the two or more functionalities.

Non-limiting examples of such materials to which the functionalities are associated include aliphatic materials, aromatic materials, carbocyclic materials, siloxy materials, sugars, amino acids, nucleic acids and others. Each of these may be substituted to modify functionality and association by one or more functional groups such as alkyl groups, aromatic groups, double or triple bonds, hydroxyl groups and other oxygen-containing groups, thiols and other sulfur-containing groups, amine groups and other nitrogen containing groups, carboxyl groups such as carboxylic acids, aldehydes, amides and others, oxide groups, silicon atoms and silicon-containing groups and others.

In some embodiments, the diagnostic tool of the invention, used in methods of the invention, as disclosed herein, may be of the structure A-L-B, wherein A is a virus-binding functionality or functionalities, B is an XRF-marker or markers, L is a linking bond (e.g., a covalent bond) or a linking atom or a group of atoms associating A and B. each of A and B may be selected as above.

Group L, being a bond or a linking atom or group of atoms, may be a bond, in which case A and B are associated directly with each other. Where L is an atom or a group of atoms, it may be selected amongst carbon-based groups, such as aliphatic groups, cyclic groups, groups containing one or more double or triple double binds; aromatic groups; heteroaromatic groups; carbocyclic groups; silicon-based groups; sugars; amino acids; nucleic acids and others.

A diagnostic tool may be one or more such materials which differ from each other in structure or identity of any of the functionalities. Different tools may be used to detect different types of virus in a sample or to quantitatively determine the relative population of one virus as compared to the other.

The invention further provide a formulation comprising a diagnostic tool and a carrier. The formulation may further comprise one or more additives selected from stabilizers, antioxidants, oxidizing materials, surfactants, lysing agents, sugars, salts, pH stabilizers, buffers, detergents, diluents, preservatives, solubilizers, emulsifiers and others.

The invention further provides a kit comprising a diagnostic tool according to the invention and instructions of use.

The invention further provides a method of determining the presence of a virus or a viral infection in a biological sample, the method comprising treating a biological sample with at least one diagnostic tool according to the invention, permitting association between the diagnostic tool and a virus, any component thereof or any antibody, substance or cell related to an immunogenic response thereto, and irradiating said sample with x-ray or gamma-ray radiation to thereby determine presence of the virus or the viral infection.

In some embodiments, the method further comprises obtaining a diagnostic tool or a solution comprising same.

In some embodiments, association between the tool and the virus/viral component/immune system component is allowed to proceed at room temperature, for an incubation time of several minutes to several hours.

In some embodiments, the method comprises a step of washing the sample to remove excess reagents, including an excess of the diagnostic tool.

In some embodiments, the method comprises a step of cell lysis.

In some embodiments, the method comprises separating between viral population that is associated to the diagnostic tool from free viral population. Such a separation may be achieved by any means known in the art, such as filtration, gravitational separation, centrifugation and others.

In some embodiments, the method comprises detection of an x-ray signal received from said sample in response to the x-ray or gamma-ray radiation and processing the signal. The signal may be read by an XRF analyzer (reader) which may include an emitter (e.g. X-ray tube) for emitting X-ray or Gamma-ray signal towards the sample and a detector for detecting the response signal arriving from the sample. Such an XRF analyzer is described in International Patent Application PCT/IL2017/051050 or any US applications derived therefrom, which are incorporated herein by reference.

The method may further comprise detecting an x-ray signal arriving from the sample in response to the x-ray or gamma-ray radiation; processing the detected response x-ray signal, to determine presence of the virus.

According to some embodiments of the invention, the processing includes filtering the detected response x-ray signal to obtain an enhanced response signal having an improved signal to noise (SNR) and/or improved signal to clutter (SNC) as compared to the originally detected response x-ray signal. Thereafter, the enhanced response signal is processed to determine whether it includes a signal and/or auxiliary signals relating to the XRF-marker which are associated with the presence of the virus. For instance, the processing may include analyzing the intensity of the response signal at one or more frequencies associated with the marking and/or auxiliary signals and thereby authenticating the object by determining if the response signal includes marking and/or auxiliary signals. The analysis may include for example performing spectral analysis to determine the spectra of the response signal in a certain frequency band overlapping with the frequencies of said marking and/or auxiliary signals, and/or is may be specifically designed to detect/determine the intensity of the response signal at the specific one or more frequencies of the marking and/or auxiliary signals.

Examples of such a filtering technique, which can be used to obtain enhanced response signal with high enough SNR and/or SCR are described for example in U.S. Provisional Pat. Application No. 62/142,100 and/or WO 2016/157185, and/or applications claiming priority therefrom, being herein incorporated by reference.

More specifically, according to some embodiments of the present invention the filtering is performed by applying a time series analysis technique to at least a portion of the wavelength spectral profile of the detected X-Ray response signal to suppress trend and/or periodic components from the wavelength spectral profile. The trend and periodic components, which are suppressed by the filtering, are associated with at least one of clutter and noise appearing in the detected portion of the X-Ray signal and sourced from one or more of the following: instrumental noise of the detection device, one or more foreign materials in the vicinity of the object, backscattering noise, and interfering signals from neighboring peaks. Therefore, the enhanced response signal in which trend and/or periodic components in the spectrum are suppressed has higher SNR and/or higher SCR as compared to the detected X-Ray response signal which allows identification of the weak marking and/or auxiliary signals therein.

In some embodiments, the diagnostic tool of the present invention may be incorporated in system for recording the results obtained by one or more systems using the diagnostic tools such that, for example, tests for the presence of a virus carried out in many different locations (possibly simultaneously) can be stored, analyzed, and/or made public in a short span of time. For that purpose, the device reading the marking (e.g. an XRF analyzer) may communicate with the database system. The database system may be on-premises, cloud-based system or a distributed ledger. In an example, the database system may be a distributed blockchain system wherein a plurality of parties store and access the relevant data (for example, health facilities and officials, hospitals, governmental and security agencies, the examined individuals, and/or the general public) . In such a blockchain system a plurality of parties may store and access data wherein the data stored is immutable, easily verifiable and, due the distributed design, inherently resistant to modification.

### Additional Specific Embodiments

In numerous embodiments the tool of the invention is applied to the biological sample that is a blood sample, a nasal excretion, an oral excretion, a urine sample or tear drops.

In certain embodiments the tool of the invention is applied to a virus selected from Coronaviridae/Corona-Virus, Orthomyxoviridae, Paramyxoviridae, Coxsackie family of viruses and Adenoviridae family.

In further embodiments the tool of the invention is applied to the causative virus of COVID-19, SARS-CoV-2.

In certain embodiments the tool of the invention is selected to associate to a region of the virus comprising a functionality selected from an amino acid, a peptide, a protein, a lipid, a carbohydrate, a nucleic acid and native functionalities.

In further embodiments the region is a region of the virus membrane, the virus capsid or virus nucleocapside.

In certain embodiments a virus-binding functionality selected from amines such as primary, secondary and tertiary amines; sulfhydryl group, carboxylic acid and derivatives thereof; alcohols, dialcohols and higher homologs thereof; thiol, dithiols and higher homologs thereof; thio esters; nitrgo; ethers; di sulfide group.

In certain embodiments the XRF marker is an atom or a group of atoms emitting an X-Ray signal in response to interrogation (irradiation) by X-Ray or gamma ray radiation.

In further embodiments the atom or group of atoms is in a form of a metal ion, an organometallic functionality, a metal oxide functionality or a non-metal atom.

In certain embodiments wherein the XRF-marker is an element or a functional group comprising an element selected from Si, P, S, Cl, K, Ca, Br, Ti, Fe, V, Cr, Mn, Co, Ni, Ga, As, Fe, Cu, Zn, Ga, Rb, Sr, Y, Zr, Nb, Mo, Tc, Ru, Rh, Ag, Cd, In, Sn, Sb, Te, I, Cs, Ba, La, Ta, W, Se and Ce.

In numerous embodiments the diagnostic tool of the invention has a structure A-L-B, wherein A is a virus-binding functionality or functionalities, B is an XRF-marker or markers, and L is a linking bond or a linking atom or a group of atoms associating A and B.

In another aspect the invention provides a formulation comprising a diagnostic tool as above and a carrier.

In numerous embodiments the formulations of the invention further comprise one or more additives selected from stabilizers, antioxidants, oxidizing materials, surfactants, lysing agents, sugars, salts, pH stabilizers, buffers, detergents, diluents, preservatives, solubilizers and emulsifiers.

In yet another aspect the invention provides a kit comprising the diagnostic tool of the invention as detailed above and instructions for use.

Still from another aspect the invention provides a method of determining the presence of a virus in a biological sample, the method comprising treating a biological sample with at least one diagnostic tool of the invention as detailed above so as to permit association between the diagnostic tool and a virus, and irradiating said sample with X-ray or gamma-ray radiation to thereby determine presence of the virus.

In certain embodiments the method of the invention comprises obtaining a diagnostic tool or a solution comprising same.

In numerous embodiments the method of the invention comprises association between the tool and the virus at room temperature for an incubation time for several minutes and up to several hours.

In certain embodiments the method of the invention comprises a step of washing the sample to remove excess reagents, including an excess of the diagnostic tool.

In further embodiments the method of the invention comprises a step of cell lysis.

In certain embodiments the method of the invention comprises separating between viral population that is associated to the diagnostic tool from free viral population.

In numerous embodiments the method of the invention comprises detecting an X-ray signal received from said sample in response to the X-ray or gamma-ray radiation and processing the signal.

1-56. (canceled)

57. A diagnostic platform for detecting a biological pathogen in a sample, the platform comprising at least one XRF identifiable marker operably linked to at least one functional component for detecting the biological pathogen or a part thereof, wherein the functional component enables marking the pathogen in the sample with the at least one XRF identifiable marker.

58. The diagnostic platform of claim 57, wherein the at least one XRF identifiable marker and the at least one functional component are operably linked by at least one non-specific (multifunctional) component.

59. The diagnostic platform of claim 57, wherein the at least one functional component is a component of a polymerase chain reaction (PCR), a reverse transcription, a nucleic acid hybridization, an antibody test, a serological antibody test.

60. The diagnostic platform of claim 57, wherein the at least one functional component is a nucleotide, an oligonucleotide, an amino acid, a peptide, a derivative, a modification, a fusion construct, a conjugate thereof.

61. The diagnostic platform of claim 57, wherein the at least one functional component is a primary of a secondary antibody.

62. The diagnostic platform of claim 57, wherein the biological pathogen is a viral or a bacterial pathogen in a mammalian disease.

63. The diagnostic platform of claim 57, wherein the sample is a biological sample obtained from a mammal at risk of being infected or already infected with the biological pathogen.

64. The diagnostic platform of claim 62, wherein the viral or the bacterial pathogen is selected from the pathogens of Cholera, Plague and Yellow Fever, Severe Acute Respiratory Syndrome (SARS), Ebola, Zika, Middle East Respiratory Ryndrome (MERS), Human Immunodeficiency Virus (HIV/AIDS), Influenza A (H1N1)pdm/09, Tuberculosis (TB) and Corona Virus Disease 19 (COVID-19).

65. The diagnostic platform of claim 63, wherein the biological sample is a sample of a body fluid, a body discharge, a body secretion obtained from a mammal at risk of being infected or already infected with the biological pathogen.

66. A composition comprising at least one XRF identifiable marker operably linked to at least one functional component for detecting a biological pathogen or a part thereof, for marking the pathogen with the at least one XRF identifiable marker that is detectable by an XRF reading device.

67. The composition of claim 66, wherein the at least one XRF identifiable marker and the at least one functional component are operably linked by at least one non-specific (multifunctional) component.

68. A method for detecting a biological pathogen in a sample, the method comprises contacting the sample with at least one XRF identifiable marker operably linked to at least one functional component for detecting the biological pathogen or a part thereof, thereby marking the pathogen in the sample with the at least one XRF identifiable marker that is detectable by an XRF reading device.

69. The method according to claim 68, comprising irradiating said sample with X-ray or gamma-ray radiation to thereby determine presence of the pathogen.

70. The method according to claim 68, for determining presence of a virus in the sample, the method comprising irradiating the sample with X-ray or gamma-ray radiation to thereby determine presence of the virus.

71. The method of claim 68, wherein the at least one XRF identifiable marker, the at least one functional component and/or at least one non-specific (multifunctional) component are operably linked by covalent bond.

72. The method of claim 68, wherein the biological pathogen is a viral or a bacterial pathogen in a mammalian disease.

73. The method of claim 68, wherein the mammalian disease is a human disease.

74. The method of claim 68, wherein the sample is a biological sample obtained from a mammal at risk of being infected or already infected with the biological pathogen.

75. The method of claim 68, wherein the sample is a non-biological sample obtained from a surface, an object, or a part thereof, which was in contact or is suspected to be in contact with a mammal at risk of being infected or already infected with the biological pathogen.

76. The method of claim 72, wherein the viral or the bacterial pathogen is selected from the pathogens of Cholera, Plague and Yellow Fever, Severe Acute Respiratory Syndrome (SARS), Ebola, Zika, Middle East Respiratory Ryndrome (MERS), Human Immunodeficiency Virus (HIV/AIDS), Influenza A (H1N1)pdm/09, Tuberculosis (TB) and Corona Virus Disease 19 (COVID-19).

77. The method of claim 74, wherein the biological sample is a sample of a body fluid, a body discharge, a body secretion obtained from a mammal at risk of being infected or already infected with the biological pathogen.

78. The method of claim 77, wherein the biological sample is a sample of blood, serum, saliva, a sample of nasal or lung discharge, urine or fecal sample or a sample of tear drops.
